(19) United States
(12) Patent Application Publication
(10) Pub. No.: US 2007/0128656 A1
(43) Pub. Date: Jun. 7, 2007

(54) Direct Fluorescent Label Incorporation Via 1st Strand cDNA Synthesis

(75) Inventors: Deepak Agrawal, Tampa, FL (US); William W. Gross, Dade City, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 11/674,714

(22) Filed: Feb. 14, 2007

Related U.S. Application Data
(62) Division of application No. 10/710,232, filed on Jun. 28, 2004.
(60) Provisional application No. 60/481,029, filed on Jun. 26, 2003.

(51) Int. Cl. C12Q 1/68; C12P 19/34; C07H 21/04

(52) U.S. Cl. 435/6; 435/91.2; 536/25.32

(57) ABSTRACT

The present invention provides for highly efficient fluorescent label incorporation during 1^{st} strand complimentary DNA (cDNA) synthesis. This is achieved by the use of labeled random primers (including, but not limited to, random hexamers, septamers, octamers, nonamers and decamers) rather than labeled nucleotides. This invention thus bypasses the well known problems of enzyme deactivation and inefficient label incorporation when using only labeled nucleotides.

### CROSS-REFERENCE TO RELATED DISCLOSURE

This disclosure is a divisional application claiming the benefit of the filing date of pending U.S. patent application entitled: “Direct Fluorescent Label Incorporation Via 1^{st} Strand cDNA Synthesis,” by the same inventor, filed on Jun. 28, 2004, bearing Ser. No. 10/710,232 which claims priority from a provisional application filed Jun. 26, 2003 by the present inventors and bearing application No. 60/481,029.

### STATEMENT OF GOVERNMENT INTEREST

This invention was made with Government support under Grant No. 1RO1CA89301 awarded by the National Cancer Institute. The Government has certain rights in the invention.

### BACKGROUND OF THE INVENTION

The process of labeling gene sequences with fluorescent labels has generally comprised a method whereby a fluorescent dye (such as cyanine 3 or cyanine 5) is attached to a complimentary strand of the target template. The target strand to be sequenced is commonly contained within a plasmid or other cloning vector. A primer that will specifically anneal to one of the 3′ ends of the template strand is chemically synthesized. Primers are generally short (15-20 base pair long), single stranded oligonucleotide sequences. When dealing with DNA the two stands of the template DNA are denatured to single strands by heat and the primer molecules bind to their complimentary sequence on the desired template strand as the mixture cools. A polymerase is then added together with a mixture of four deoxyribonucleotides triphosphates (dNTPs). The four dNTPs include dATP, dCTP, dGTP and dTTP. Each dNTP can be coupled with a fluorescent dye, although this most commonly occurs with dCTP. The primed complexes are then extended by the polymerase toward their 3′ ends by random polymerization of each of the nucleotides from the dNTP pool.

### SUMMARY OF INVENTION

An improved method for the incorporation of fluorescent label tags during first strand cDNA synthesis. This method can be used in any test or assay requiring fluorescent tags for qualitative or quantitative measurement in molecular biology processes where a DNA, RNA or oligonucleotide target of any length is identified via a hybridization reaction to a fluorescently labeled probe. The method is particularly applicable to cDNA microarray experiments where such targets as oligonucleotide sequences are probed and identified by successfully hybridizing them with complementary fluorescently labeled probe sequences.

The present invention provides for highly efficient fluorescent label incorporation during 1^{st} strand complimentary DNA (cDNA) synthesis. This is achieved by the use of labeled random primers (including, but not limited to, random hexamers, septamers, octamers, nonamers and decamers) rather than labeled nucleotides. This invention thus bypasses the well known problems of enzyme deactivation and inefficient label incorporation when using only labeled nucleotides.

As an improved method for the incorporation of fluorescent label tags during first strand cDNA synthesis, this method can be used in any test or assay requiring fluorescent tags for qualitative or quantitative measurement in molecular biology processes where a DNA, RNA or oligonucleotide target of any length is identified via a hybridization reaction to a fluorescently labeled probe. The method is particularly applicable to cDNA microarray experiments where such targets as oligonucleotide sequences are probed and identified by successfully hybridizing them with complementary fluorescently labeled probe sequences.

An advantage of the inventive method is that each complimentary strand (DNA or RNA) produced reaches the proper terminal length. Each strand is the same length as the original total RNA template.

Another advantage of the present invention is that each complimentary (DNA or RNA) has a fixed, and known, number of fluorescent tags attached to it, via the primer. This allows a more exact quantification in any experiment where the fluorescently labeled strand will be detected with appropriate instrumentation.

Still another advantage of the inventive method is that any length primer can be used, including random oilgos (i.e. pentamers, hexamers, septamers, octamers, nonamers and decamers). Oligo(dT), of any length, may also be specified (although most oligo(dT) molecules are 12 to 20 bases long). Gene specific primers (GSP) may also be modified for use in the inventive method.

Another advantage is that a quenching step of the prior art is no longer necessary. In the present method the ehylenediaminetetraacetic acid (EDTA) will chelate, or bind, some of the divalent ions, such as magnesium, required by the enzyme to function. Without these ions, the enzyme ceases to function.

### BRIEF DESCRIPTION OF THE DRAWINGS

For a fuller understanding of the nature and objects of the invention, reference should be made to the following detailed description, taken in connection with the accompanying drawings, in which:

FIG. 1 is a diagrammatic representation of the prior art.

FIG. 2 is a diagrammatic representation of the inventive method.

FIG. 3 is a diagrammatic representation of the deficiencies of the prior art, specifically how fluorescent labels can interfere with the binding process when attached to individual dNTP molecules.

FIG. 4 is a diagrammatic representation of the inventive method showing that when the fluorescent label is attached to the primer, there is no interference with the attachment of the individual dNTP molecules.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

Many techniques in molecular biology, such as micro-array experiments, depend upon identifying the presence/abundance of a known/unknown RNA/DNA strand of some length. A fluorescent label is attached to a “target” strand in a living cell or in an extract of a living cell. The fluorescent label is then detected after hybridization has occurred thus verifying the presence of the target in the cell/cell extract. As an example, this method can be used in flow cytometry type apparatus to separate the labeled product from unlabeled product in a research or industrial application, since the hybridized strands can be easily separated later for collection.

For “typical” micro-array experiments, it is necessary to generate the first strand of cDNA, which if done with labeled primers (rather than individual dNTP molecules) will yield a quality assurance measure of the quantity of transcript that has been generated. This transcript (the cDNA product) is then subjected to a second round of polymerization to generate a strand that is congruent to (exactly the same as) the original strand of RNA.

First Strand Synthesis

First strand synthesis makes a strand of cDNA using a strand, or more usually multiple strands of poly(A)+ RNA template. The typical protocol for such a reaction (without fluorescent labels) is as follows:

Components Required

1 nanogram to 10 micrograms of total RNA isolated from plant or animal cells

or

1 nanogram to 500 nanograms of mRNA (messenger RNA or poly(A)+ RNA)

30 to 200 units of a reverse transcriptase (an enzyme known as RNA dependent DNA polymerase) depending upon manufacturer's recommendation

0.5 micrograms of oligo(dT) primer

or

50 to 250 nanograms of random primers (hexamers, septamers, octamers, etc.)

10 millimoles of dNTP mix (10 millimoles each dATP, dGTP, dCTP, dTTP)

distilled water

5× or 10× first strand buffer (necessary for enzyme activation)

0.1 Molar Dithiothreitol (DTT)

1^{st} Strand Synthesis Protocol

0.5 microgram of oligo(dT) primer (or up to 250 nanograms of random primer), up to 10 micrograms of total RNA (or up to 500 nanograms of mRNA) and 1 microliter of 10 milliMole of dNTP mix is combined with up to 20 microliters of distilled water and heated to 65 degrees centigrade for 5 minutes and then chilled on ice for about 1 minute.

To this mixture is then added approximately 5 microliters of 5× or 10× first strand buffer (amount and concentration varies with enzyme used), 2 microliters of DTT and approximately 30 to 200 units of the desired reverse transcriptase.

The mixture is then incubated for 1 to 2 hours at 42 to 50 degrees centigrade.

At the end of the incubation period, the enzyme is quenched by adding ethylenediaminetetraacetic acid (EDTA) and the RNA template is either degraded or digested so that the synthesized 1^{st} strand cDNA can be extracted via various purification steps.

Typical Direct Labeling Reaction

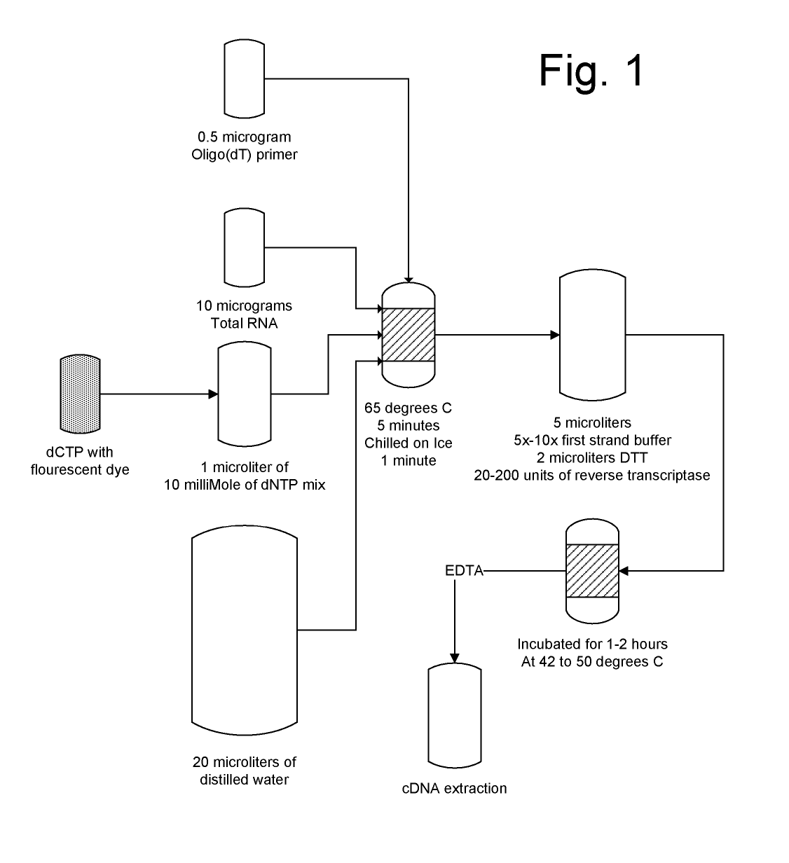

The typical protocol for producing 1^{st} strand cDNA with fluorescent label incorporation, as shown in FIG. 1, is essentially the same as the above enumerated 1^{st} strand synthesis, except for the dNTP mixture. In this case, one of the nucleotides of this mixture (usually dCTP) will be supplied with a fluorescent dye (usually cyanine 3 or cyanine 5) attached to it chemically. During the enzymatic reaction, it is believed that the enzyme will incorporate this labeled dCTP when producing the complementary strand.

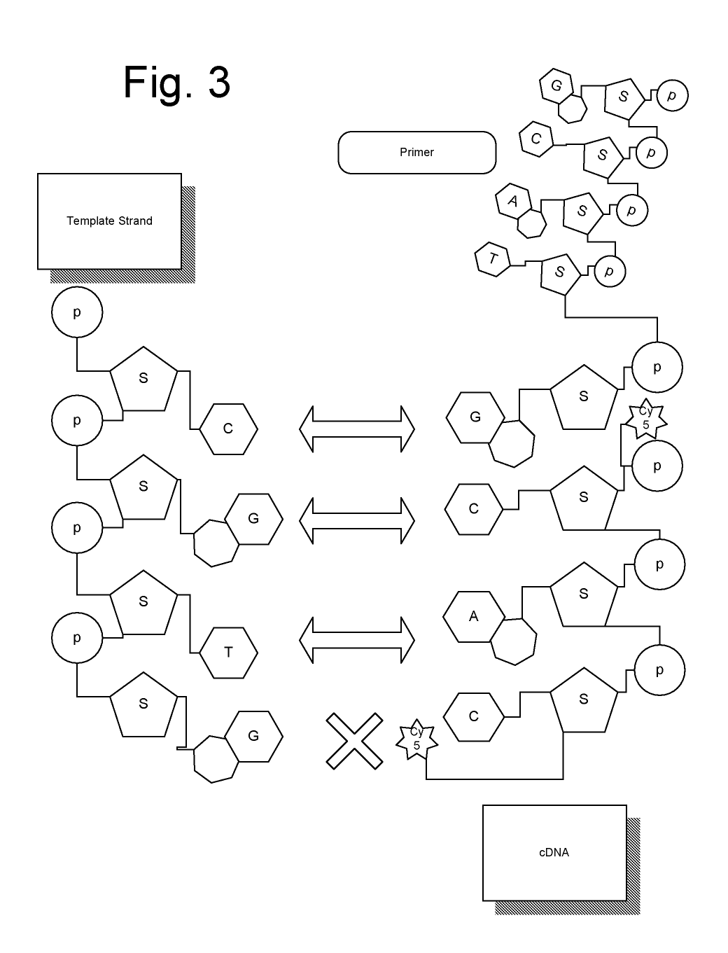

The prior methods used an unlabeled primer (no fluorescent tag). When the primer is introduced into the total RNA/chemical cocktail, the primer attaches itself to the total RNA stand and is lengthened by the polymerase. That is to say that the polymerase begins to add individual dNTP molecules to one end of the primer to make a complimentary DNA (cDNA) strand copy of the original total RNA strand. During this process it is expected that the polymerase will incorporate, or add to the primer, one or more of the fluorescently labeled dNTP molecules. Very often this process is ineffective, as shown in FIG. 3, since the labeled dNTP molecules can inhibit, or quench, the polymerase preventing completion of the strand. As a result, a shorter cDNA strand is formed that contains few or no fluorescent labels.

Another consequence of employing this method is that each synthesized strand of cDNA will have unknown number, fewer or greater, of fluorescent dNTP molecules incorporated into the new strand depending on the base sequence of the total RNA that was used as a template.

New Method Of Primer Labeling

The method described here uses a fluorescent dye (such as cyanine 3 or cyanine 5) chemically attached to the primer (either random primers or the oligo(dT) primer). Labeling the primer leads to a much more efficient and robust incorporation of the fluorescent label into the synthesized cDNA without quenching the enzyme that is reported to limit the overall success of the direct incorporation reaction described above.

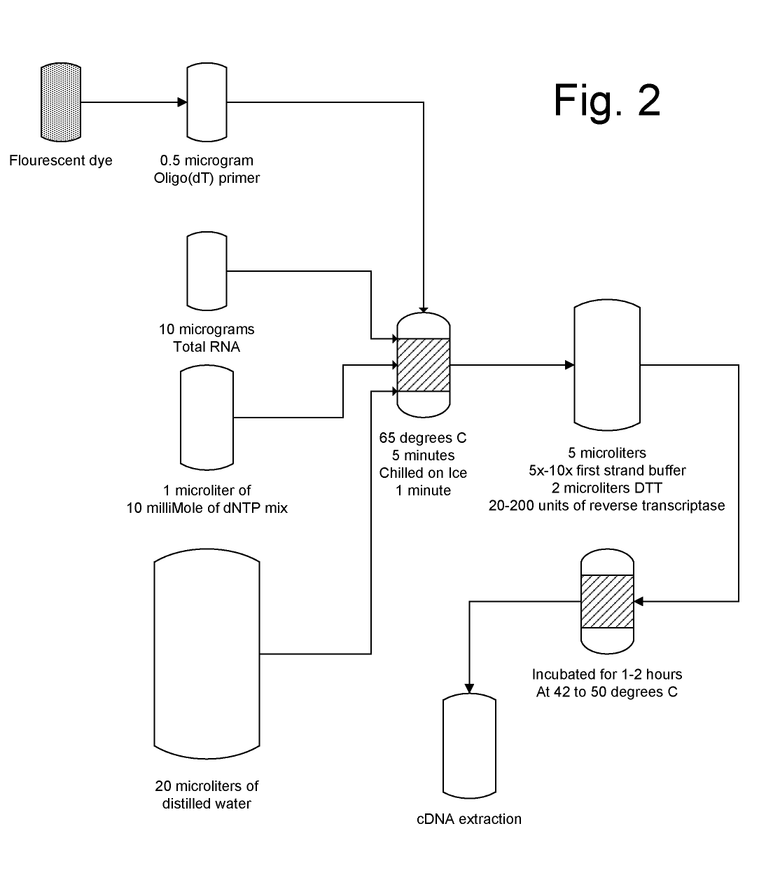

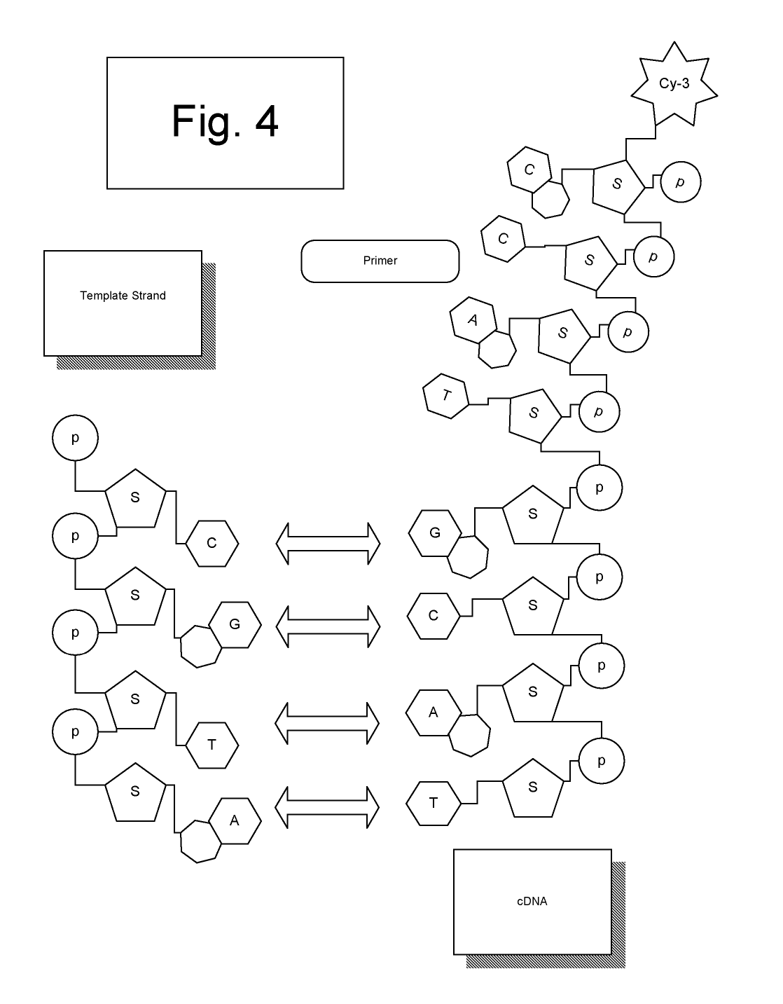

The inventive method, shown in FIG. 2, differs from the prior art in that one begins with a primer that is already labeled with one or more fluorescent labels attached to one end of the primer. In one embodiment the label(s) is attached to the end of the primer which will not be extended, the 5 prime end. After the primer has attached to the total RNA template, the polymerase incorporates the unadulterated dNTP molecules. As shown in FIG. 4, the polymerase is no longer inhibited by the fluorescent labels attached to the dNTP molecules. This method prevents the quenching of the dNTP incorporation since none of the dNTP molecules are labeled.

Use of the inventive method assures that each synthesized strand of cDNA will contain an equal fluorescent signature when stimulated. This allows a user to more accurately quantify the population of total RNA template strands in the original sample. Such an improvement provides tremendous advantages in micro-array, as well as other, experiments. In fact, any experiment where hybridization reactions occur and the measurement of the template species is required will benefit from this method.

For example, in clinical diagnostics the ongoing discoveries of how RNA interacts with proteins in cell signaling pathways requires applications that can use this technique. Such experiments require the ability to determine the relative abundance of the RNA strands in question. This technique will allow a direct quantification of said RNA in a similar fashion to the technique “FISH”, or Fluorescence In Situ Hybridization (where a labeled probe is used with a complete DNA strand with label attached). Such techniques may require the use of a GSP (gene specific primer) unless one seeks only to identify relative abundance of RNA in the cell nucleus.

It will be seen that the objects set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be the to fall therebetween. Now that the invention has been described,

What is claimed is:

1. A method of incorporating a fluorescent label during first strand complementary DNA synthesis comprising the steps of:
providing a primer with a fluorescent label attached to one end; and
synthesizing a first strand of complementary DNA.

2. The method of claim 1 wherein the primer is a random primer.

3. The method of claim 2 wherein the random primer is chosen from the group consisting of random hexamers, septamers, octamers, nonamers and decamers.

4. The method of claim 1 wherein the primer is a gene specific primer.

5. The method of claim 1 wherein the fluorescent label is cyanine 3.

6. The method of claim 1 wherein the fluorescent label is cyanine 5.

7. The method of claim 1 wherein the fluorescent label is chemically attached to the 5′ end of the primer.

8. The method of claim 1 wherein the first stand of complementary DNA is synthesized comprising the steps of;
combining the primer, a template sequence, about 1 microliter of about 10 milliMole of dNTP mix and about 1 to 20 microliters of distilled water;
heating the combination;
cooling the combination;
adding to the combination about 5 microliters of first strand buffer, about 2 microliters of DTT and about 30 to 200 units of a reverse transcriptase; and
incubating the total combination.

9. The method of claim 8 wherein the primer is a random primer.

10. The method of claim 9 wherein the amount of random primer is between about 1 and 250 nanograms.

11. The method of claim 8 wherein the template sequence is total RNA.

12. The method of claim 11 wherein the amount of total RNA is between about 1 and 10 micrograms.

13. The method of claim 8 wherein wherein the template sequence is mRNA.

14. The method of claim 13 wherein the amount of mRNA is between about 1 and 500 nanograms.

15. The method of claim 8 wherein the combination is heated at about 65 degrees centigrade.

16. The method of claim 8 wherein the combination is heated for about 5 minutes.

17. The method of claim 8 wherein the combination is cooled on ice for about 1 minute.

18. The method of claim 8 wherein the first strand buffer is about 5× first strand buffer.

19. The method of claim 18 wherein the first strand buffer is about 10× first strand buffer.

20. The method of claim 8 wherein the combination is incubated at between about 42 and 50 degrees centigrade.

21. The method of claim 8 wherein the combination is incubated between about 1 and 2 hours.

22. The method of claim 8 wherein the enzymatic activity of the combination is quenched by adding ehylenediaminetetraacetic acid.

23. The method of claim 8 wherein the synthesized first strand complementary DNA is extracted from the mixture.
